(19) United States
(12) Patent Application Publication
(10) Pub. No.: US 2023/0113699 A1
(43) Pub. Date: Apr. 13, 2023

(54) PROFILE ORIENTED COGNITIVE IMPROVEMENT SYSTEM AND METHOD

(71) Applicant: FEUERSTEIN LEARNING AND THINKING, LTD., Jerusalem (IL)

(72) Inventor: Refael FEUERSTEIN, Jerusalem (IL)

(21) Appl. No.: 17/766,247

(22) PCT Filed: Sep. 29, 2020
(86) PCT No.: PCT/IL2020/051061

§ 371 Date: Apr. 3, 2022

Related U.S. Application Data
(60) Provisional application No. 62/909,295, filed on Oct. 2, 2019.

(51) Int. Cl. G16H 20/70; G16H 50/20

(52) U.S. Cl. CPC G16H 20/70; G16H 50/20

(57) ABSTRACT

A profile oriented cognitive improvement method, constituted of: outputting a plurality of diagnostic exercises; for each of the output diagnostic exercises, receiving a respective input; determining whether the respective received input is within respective parameter limits associated with at least one of a plurality of predetermined error types; responsive to the determination, identifying the at least one error type; comparing a function of the identified error types to each of a plurality of models; responsive to an outcome of the comparison, identifying which of a plurality of field profiles describes the user; and outputting a respective portion of a respective set of improvement exercises associated with the identified field profile.

### CROSS-REFERENCE TO RELATED APPLICATIONS

The present application claims priority from U.S. provisional patent application Ser. 62/909,295, filed Oct. 2, 2019, and entitled “PROFILE ORIENTED COGNITIVE IMPROVEMENT SYSTEM AND METHOD”, the entire contents of which incorporated herein by reference.

### TECHNICAL FIELD

The invention relates generally to the field of computerized cognitive improvement systems, and in particular to a profile oriented cognitive improvement system and method.

### BACKGROUND

Cognitive skills play a crucial role in the daily functioning of people. Individuals are born with a certain level of cognitive skills, and these skills are further developed during schooling and other activities. However, the cognitive skills developed in each individual may be different, and as a result some individuals may find that their cognitive skills in certain areas may be at a level which interferes with daily functioning. Furthermore, some of these cognitive skills (e.g., memory, problem-solving activities, or speed processing) decline in the process of aging. Suggested methods of intervention for slowing the process of cognitive decline include: medication; specific diets; physical exercise; music therapy; and cognitive exercises, such as cross-word puzzles. Unfortunately, none of these methods have shown proven significant improvement in cognitive abilities.

### SUMMARY OF THE INVENTION

Accordingly, it is a principal object of the present invention to overcome disadvantages of prior art cognitive improvement systems. This is provided in one embodiment by a profile oriented cognitive improvement system comprising: a processor; an output device arranged to provide a user with visual, audio or printed output responsive to the processor; an input device arranged to receive a user input; and a memory having stored thereon: data regarding a plurality of diagnostic exercises, each of the plurality of diagnostic exercises having associated therewith, for each of a plurality of predetermined error types, respective parameter limits indicative of the respective error type; data regarding a plurality of models, each of the plurality of models associated with a respective one of a plurality of field profiles, each of the plurality of field profiles associated with a respective one of the plurality of predetermined error types; and data regarding a plurality of improvement exercises, each of a plurality of sets of the plurality of improvement exercises associated with a respective one of the plurality of field profiles; wherein the processor is arranged to: control the output device to output, in a first predetermined sequence, the plurality of diagnostic exercises, each of the plurality of diagnostic exercises prompting a user to input a response at the input device; for each of the output diagnostic exercises, receive a respective input at the input device; for each of the received inputs, determine whether the respective received input is within the predetermined parameter limits of the respective diagnostic exercise associated with at least one of the plurality of predetermined error types; for each of the received inputs, responsive to the determination that the respective received input is within the predetermined parameter limits associated with the at least one of the plurality of predetermined error types, identify the at least one of the plurality of predetermined error types; for each of the received inputs, responsive to the identification, store an indication of the identified at least one of the plurality of predetermined error types on the memory; apply a predetermined first function to the indications of the identified error types for the received inputs; compare an output of the applied predetermined first function to each of the plurality of models stored on the memory; responsive to an outcome of the comparison, identify which of the plurality of field profiles describes the user; control the output device to output, in a second predetermined sequence, a respective portion of the respective set of the plurality of improvement exercises associated with the identified field profile, each of the plurality of improvement exercises prompting a user to input a response at the input device.

In one embodiment, the plurality of predetermined error types comprises: input error type; output error type; and processing error type. In another embodiment, the processor is further arranged to: for each of the predetermined error types, determine the number of the received inputs having the respective error type; for each of the predetermined error types, compare the determined number to a respective predetermined error threshold value; and for each of the predetermined error types, responsive to an output of the comparison to the respective predetermined error threshold value, determine whether the user exhibits the respective error type, wherein the applied predetermined first function comprises the determination of the error type exhibition of the user, and wherein each of the plurality of models comprises a respective combination of the predetermined error types.

In one embodiment, the processor is further arranged to: for each of the received inputs, associated with a respective one of the output diagnostic exercises, and responsive to a determination that the respective received input is not within the respective predetermined parameter limits associated with any of the plurality of predetermined error types, store on the memory an indication of the determination; responsive to the stored indications of the determinations, determine a predetermined accuracy value of a percentage of correct inputs associated with the output diagnostic exercises; and responsive to the determined accuracy value, determines a level of difficulty of the portion of the output improvement exercises. In one further embodiment, the data regarding each of the plurality of improvement exercises comprises, for each of the plurality of predetermined error types, respective parameter limits indicative of the respective error type, and wherein the processor is further arranged to: for each of the output improvement exercises, receive a respective input at the input device; for each of the received inputs, associated with a respective output improvement exercise, and responsive to a determination that the respective received input is not within the respective predetermined parameter limits associated with any of the plurality of predetermined error types, store on the memory an indication of the determination; responsive to the stored indications of the determinations, associated with the output improvement exercises, determine the accuracy value of a percentage of correct inputs associated with the output improvement exercises; compare an outcome of the accuracy value of the percentage of correct inputs associated with the output improvement exercises with the outcome of the accuracy value of the percentage of correct inputs associated with the output diagnostic exercises; and output an outcome of the accuracy value outcome comparison.

In one embodiment, responsive to the determined accuracy value of the percentage of correct input associated with the output diagnostic exercises, the processor is further arranged to control a pace of the output improvement exercises. In another embodiment, the processor is further arranged to: for each of the output diagnostic exercises, determine the amount of time which passed from the respective output to the respective received input; determine a predetermined time function to the determined amounts of time; and control a pace of the output improvement exercises responsive to the determined time function.

In one embodiment, the data regarding each of the plurality of improvement exercises comprises, for each of the plurality of predetermined error types, respective parameter limits indicative of the respective error type, and wherein the processor is further arranged to: for each of the output improvement exercises, receive a respective input at the input device; for each of the received inputs, associated with a respective improvement exercise, determine whether the respective received input is within the predetermined parameter limits of the respective improvement exercise associated with at least one of the plurality of predetermined error types; for each of the received inputs, associated with a respective improvement exercise, and responsive to the determination that the respective received input is within the predetermined parameter limits associated with the at least one of the plurality of predetermined error types, identify the at least one of the plurality of predetermined error types; for each of the received inputs, associated with a respective improvement exercise, responsive to the error type identification, store an indication of the identified at least one of the plurality of predetermined error types on the memory; apply the predetermined first function to the indications of the identified error types for the received inputs associated with the respective output improvement exercises; compare an output of the applied predetermined first function, associated with the respective output improvement exercises, to each of the plurality of models stored on the memory; responsive to an outcome of the comparison, associated with the respective output improvement exercises, identify which of the plurality of field profiles describes the user; compare the identified field profile of the user, associated with the output improvement exercises, with the identified field profile of the user associated with the output diagnostic exercises; and control the output device to output a respective portion of a respective set of the plurality of improvement exercises, the respective portion of the respective set responsive to an outcome of the comparison of the identified field profiles. In one further embodiment, the processor is further arranged to output an outcome of the comparison of the identified field profiles.

In one embodiment, the plurality of diagnostic exercises comprises a plurality of groups of diagnostic exercises, each of the plurality of groups associated with a respective one of a plurality of cognitive fields, the field profile identified separately for each of the plurality of cognitive fields, wherein the plurality of sets of improvement exercises comprises a plurality of groups of sets of improvement exercises, each of the plurality of groups associated with a respective one of the plurality of cognitive fields, the output of the improvement exercises being for each of the plurality of cognitive fields. In another embodiment, the processor is further arranged to identify, responsive to the identified field profile of each of the plurality of cognitive fields, a common profile which described the user, the identified common profile responsive to a predetermined common profile function of the identified field profiles of the plurality of cognitive fields.

In one independent embodiment, a profile oriented cognitive improvement method is provided, the method comprising: storing on a memory data regarding a plurality of diagnostic exercises, each of the plurality of diagnostic exercises having associated therewith, for each of a plurality of predetermined error types, respective parameter limits indicative of the respective error type; storing on the memory data regarding a plurality of models, each of the plurality of models associated with a respective one of a plurality of field profiles, each of the plurality of field profiles associated with a respective one of the plurality of predetermined error types; storing on the memory data regarding a plurality of improvement exercises, each of a plurality of sets of the plurality of improvement exercises associated with a respective one of the plurality of field profiles; controlling an output device to output, in a first predetermined sequence, the plurality of diagnostic exercises, each of the plurality of diagnostic exercises prompting a user to input a response at an input device; for each of the output diagnostic exercises, receiving a respective input at the input device; for each of the received inputs, determining whether the respective received input is within the predetermined parameter limits of the respective diagnostic exercise associated with at least one of the plurality of predetermined error types; for each of the received inputs, responsive to the determination that the respective received input is within the predetermined parameter limits associated with the at least one of the plurality of predetermined error types, identifying the at least one of the plurality of predetermined error types; for each of the received inputs, responsive to the identification, storing an indication of the identified at least one of the plurality of predetermined error types on the memory; applying a predetermined first function to the indications of the identified error types for the received inputs; comparing an output of the applied predetermined first function to each of the plurality of models stored on the memory; responsive to an outcome of the comparison, identifying which of the plurality of field profiles describes the user; controlling the output device to output, in a second predetermined sequence, a respective portion of the respective set of the plurality of improvement exercises associated with the identified field profile, each of the plurality of improvement exercises prompting a user to input a response at the input device.

In one embodiment, the plurality of predetermined error types comprises: input error type; output error type; and processing error type. In another embodiment, the method further comprises: for each of the predetermined error types, determining the number of the received inputs having the respective error type; for each of the predetermined error types, comparing the determined number to a respective predetermined error threshold value; and for each of the predetermined error types, responsive to an output of the comparison to the respective predetermined error threshold value, determining whether the user exhibits the respective error type, wherein the applied predetermined first function comprises the determination of the error type exhibition of the user, and wherein each of the plurality of models comprises a respective combination of the predetermined error types. In another embodiment, the method further comprises: for each of the received inputs, associated with a respective one of the output diagnostic exercises, and responsive to a determination that the respective received input is not within the respective predetermined parameter limits associated with any of the plurality of predetermined error types, storing on the memory an indication of the determination; responsive to the stored indications of the determinations, determining a predetermined accuracy value of a percentage of correct inputs associated with the output diagnostic exercises; and responsive to the determined accuracy value, determining a level of difficulty of the portion of the output improvement exercises.

In one further embodiment, the data regarding each of the plurality of improvement exercises comprises, for each of the plurality of predetermined error types, respective parameter limits indicative of the respective error type, wherein the method further comprises: for each of the output improvement exercises, receiving a respective input at the input device; for each of the received inputs, associated with a respective output improvement exercise, and responsive to a determination that the respective received input is not within the respective predetermined parameter limits associated with any of the plurality of predetermined error types, storing on the memory an indication of the determination; responsive to the stored indications of the determinations, associated with the output improvement exercises, determining the accuracy value of a percentage of correct inputs associated with the output improvement exercises; comparing an outcome of the accuracy value of the percentage of correct inputs associated with the output improvement exercises with the outcome of the accuracy value of the percentage of correct inputs associated with the output diagnostic exercises; and outputting an outcome of the accuracy value outcome comparison. In another further embodiment, responsive to the determined accuracy value of the percentage of correct input associated with the output diagnostic exercises, the method further comprises controlling a pace of the output improvement exercises.

In one embodiment, the method further comprises: for each of the output diagnostic exercises, determining the amount of time which passed from the respective output to the respective received input; determining a predetermined time function to the determined amounts of time; and controlling a pace of the output improvement exercises responsive to the determined time function. In another embodiment, the data regarding each of the plurality of improvement exercises comprises, for each of the plurality of predetermined error types, respective parameter limits indicative of the respective error type, wherein the method further comprises: for each of the output improvement exercises, receiving a respective input at the input device; for each of the received inputs, associated with a respective improvement exercise, determining whether the respective received input is within the predetermined parameter limits of the respective improvement exercise associated with at least one of the plurality of predetermined error types; for each of the received inputs, associated with a respective improvement exercise, and responsive to the determination that the respective received input is within the predetermined parameter limits associated with the at least one of the plurality of predetermined error types, identifying the at least one of the plurality of predetermined error types; for each of the received inputs, associated with a respective improvement exercise, and responsive to the error type identification, storing an indication of the identified at least one of the plurality of predetermined error types on the memory; applying the predetermined first function to the indications of the identified error types for the received inputs associated with the respective output improvement exercises; comparing an output of the applied predetermined first function, associated with the respective output improvement exercises, to each of the plurality of models stored on the memory; responsive to an outcome of the comparison, associated with the respective output improvement exercises, identifying which of the plurality of field profiles describes the user; comparing the identified field profile of the user, associated with the output improvement exercises, with the identified field profile of the user associated with the output diagnostic exercises; and controlling the output device to output a respective portion of a respective set of the plurality of improvement exercises, the respective portion of the respective set responsive to an outcome of the comparison of the identified field profiles. In one further embodiment, the method further comprises outputting an outcome of the comparison of the identified field profiles.

In one embodiment, the plurality of diagnostic exercises comprises a plurality of groups of diagnostic exercises, each of the plurality of groups associated with a respective one of a plurality of cognitive fields, the field profile identified separately for each of the plurality of cognitive fields, and wherein the plurality of sets of improvement exercises comprises a plurality of groups of sets of improvement exercises, each of the plurality of groups associated with a respective one of the plurality of cognitive fields, the output of the improvement exercises being for each of the plurality of cognitive fields. In another embodiment, the method further comprises identifying, responsive to the identified field profile of each of the plurality of cognitive fields, a common profile which described the user, the identified common profile responsive to a predetermined common profile function of the identified field profiles of the plurality of cognitive fields.

Additional features and advantages of the invention will become apparent from the following drawings and description.

### BRIEF DESCRIPTION OF THE DRAWINGS

For a better understanding of the invention and to show how the same may be carried into effect, reference will now be made, purely by way of example, to the accompanying drawings in which like numerals designate corresponding elements or sections throughout.

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the accompanying drawings:

FIG. 1A illustrates a high level schematic diagram of a profile oriented cognitive improvement system, comprising a user station, a processor and a memory, in accordance with certain embodiments;

FIG. 1B illustrates a high level block diagram of a more detailed embodiment of the processor of the profile oriented cognitive improvement system of FIG. 1A;

FIG. 1C illustrates a high level block diagram of a more detailed embodiment of the memory of the profile oriented cognitive improvement system of FIG. 1A;

FIGS. 2A-2F illustrate various high level flow charts of a first method of operation of the profile oriented cognitive improvement system of FIGS. 1A-1C; and

FIG. 3 illustrates a high level flow chart of a second method of operation of the profile oriented cognitive improvement system of FIGS. 1A-1C.

### DETAILED DESCRIPTION OF THE PREFERRED EMBODIMENTS

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

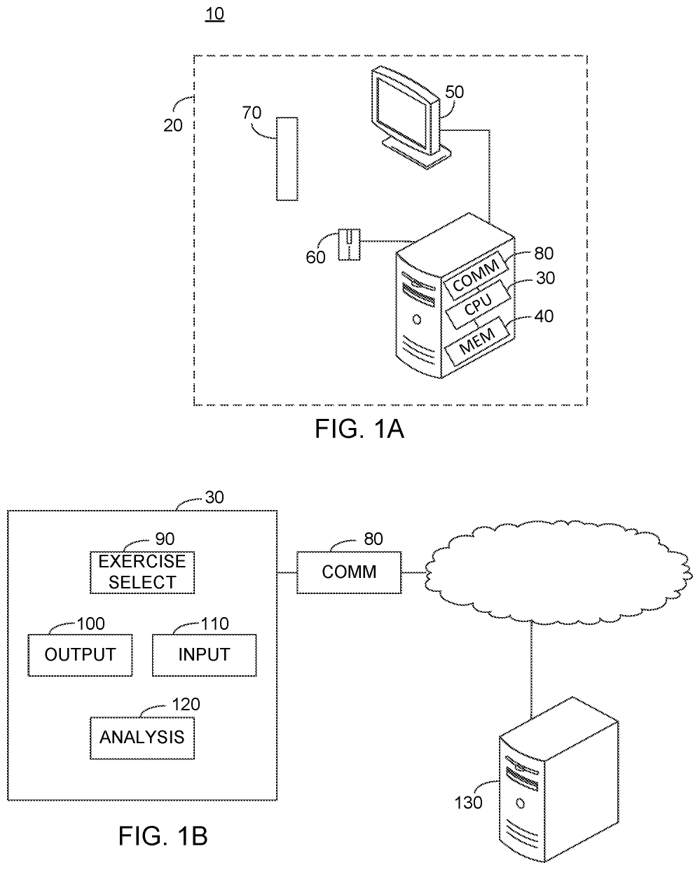

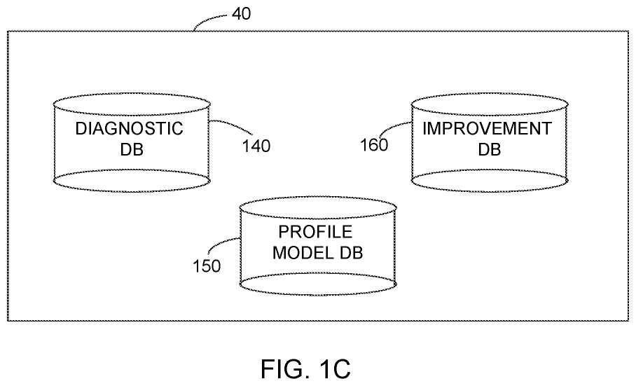

FIG. 1A illustrates a high level schematic diagram of a profile oriented cognitive improvement system 10. Profile oriented cognitive improvement system 10 comprises: a user station 20 comprising: a processor 30; a memory 40; an output device 50; an input device 60; a user support 70, such as a chair; and an optional communication module 80. Output device 50 is arranged to output data to a user. In one embodiment, output device 50 is arranged to provide a user with visual, audio and/or printed output. In one further embodiment, output device 50 comprises a computer display, speakers and/or a printer. Input device 60 is arranged to receive an input from a user at user station 20. In one embodiment, input device 60 comprises, without limitation: a mouse; a keyboard; a touch screen; and/or a voice detection system, such as a microphone in communication with a speech detection program. Processor 30 and memory 40 are illustrated as being co-located within a user work-station, computer or mobile device such as a notebook, notepad or smartphone, it being understood that each of processor 30 and memory 40 may be only partially location with the user work-station or computer, with some of the functionality located in a server in communication through communication module 80 with the user work-station, computer or mobile device.

FIG. 1B illustrates a high level block diagram of a more detailed embodiment of processor 30. Particularly, processor 30 comprises: an exercise selection module 90 in communication with memory 40; an output module 100 in communication with output device 50; an input module 110 in communication with input device 60; and an analysis module 120 in communication with memory 40. The connections between the various modules of processor 30 and between memory 40, output device 50 and input device 60 are not shown for simplicity. In one embodiment, communication module 80 is in communication with an optional server 130 and provides for bidirectional communication between processor 30 and optional server 130. In one embodiment, a portion of memory 40 is located in optional server 130, with access via communication module 80. In one embodiment, each of exercise selection module 90, output module 100, input module 110 and analysis module 120 is implemented by a portion of code stored in memory 40 when run on processor 30. In one embodiment communication module 80 is implemented as a portion of wireless local area network, a portion of mobile telephone network, or a portion of a wired local area network, without limitation.

FIG. 1C illustrates a high level block diagram of a more detailed embodiment of memory 40. Particularly, memory 40 comprises: a diagnostic exercise database 140; a profile model database 150; and an improvement exercise database 160. Diagnostic exercise database 140 has stored therein data regarding a plurality of diagnostic exercises. The data regarding the plurality of diagnostic exercises comprises sequence information, i.e. in what sequence the diagnostic exercises should be output.

The plurality of diagnostic exercises are organized in a plurality of cognitive fields, such as memory improvement, attention improvement, logical thinking, abstract thinking, spatial cognition and spatial organization, without limitation. Particularly, a first group of diagnostic exercises are associated with a first cognitive field, a second group of diagnostic exercises are associated with a second cognitive field, and so forth. As will be described below, a plurality of diagnostic tests are presented to the user, each diagnostic test comprises a respective group of diagnostic exercises. In some embodiments, at least one of the diagnostic tests may comprise a subset of the respective group of diagnostic exercises.

Each of the diagnostic exercises has associated therewith, for each of a plurality of predetermined error types, respective parameter limits indicative of the respective error type. Particularly, for each diagnostic exercise, the data includes details of when an answer is considered an error and what type of error it is, such that when the answer is within the parameter limits of a respective error type, the answer is considered an error of that respective type. In one embodiment, parameter limits of different error types partially overlap such that the answer is considered an error of more than one type.

In one embodiment, the plurality of error types comprises: an input error type; an output error type; and a processing error type. Specifically, an input error type represents errors which are caused by a problem with the ability of the user to gather data. This includes problems with correctly gathering data and problems with the process of gathering the data. Such problems can manifest, without limitation, in: reading and understanding instructions; and/or reading and understanding text displayed during the exercise. In one further embodiment, an input type error can be, without limitation: a response which answers at least mostly correctly only part of the question asked and ignores the rest of the question, or which completes at least mostly correctly only part of the exercise and ignores the rest of the exercise; a response which at least answers mostly correctly the question asked, however the order of the different details in the answer, or in the answers themselves, is incorrect; and/or the speed at which the answer was given is faster than typically possible with the respective amount of information in the question. In further detail, in the example where an input error is determined responsive to the speed of answering being too great, the parameter limits indicative of an input type error is a predetermined maximum speed for answering. Thus, if the predetermined maximum speed is exceeded, the response is determined as an input type error since it is within the respective parameter limits for a speed of response which is too fast.

In another embodiment, the details associated with the respective parameter limits of a response to a first exercise is determined in relation to a response to a second exercise. For example, if a correct response is received for a question (e.g. a mathematical equation) having a small amount of information in the question and an incorrect response is received for the same question, but having a large amount of information (e.g. the same mathematical equation, but asked as a word problem, the incorrect response is in one embodiment considered an input type error.

In one embodiment, as will be described below, the response time to a respective exercise is measured and an input error type is determined responsive to the measured response time. In another embodiment, as will be described below, a question is displayed, without displaying possible answers, and responsive to a user input the possible answers are then displayed and the question is hidden. The amount of time between display of the question until receipt of the user input request to display the possible answers is measured and the amount of time from when the possible answers are displayed until an answer was selected is also measured. An input error type is determined responsive to the outcome of a comparison between the amount of time the question was displayed and the amount of time the answers were displayed. Specifically, a larger amount of time spent on reading the question than on answering a question is stored as an indication of an input type error.

An output type error represents errors which are caused by a problem with the ability to correctly output processed information. In one further embodiment, an output type error can be, without limitation: a response which selects an answer, out of a plurality of possible answers, which is visually the most prominent; and/or a response which selects an answer which, although is incorrect, is close to the correct answer, and it has been determined that it is not an input type error.

In another embodiment, the error of a response, i.e. the details associated with the respective parameter limits, to a first exercise is determined in relation to a response to a second exercise. For example, if a correct response is received in a first exercise with a small number of displayed possible answers and an incorrect response is received to a second, similar, exercise with a larger number of displayed possible answers, the response is considered an output type error.

In another embodiment, the error type of a response is determined after a predetermined number of occurrences of the same error. For example, if in a large portion of certain exercises the answers selected were always at the bottom of the screen, these responses will be considered as an output type error.

In another embodiment, as will be described below, a question is displayed, without displaying possible answers, and responsive to a user input the possible answers are then displayed. The amount of time between display of the question until receipt of the user input request to display the possible answers is measured and the amount of time from when the possible answers are displayed until an answer was selected is also measured. An output error type is determined responsive to the outcome of a comparison between the amount of time the question was displayed and the amount of time the answers were displayed. Specifically, a larger amount of time spent on selecting the correct answer than on reading the question is stored as an indication of an output type error.

A processing type error represents errors which are caused by a problem with the user's ability to correctly process information relevant to the exercise, i.e. to correctly turn the information into knowledge. This includes, without limitation: problems in organization of data; and problems with correctly and/or efficiently creating links between different portions of the data. In one further embodiment, examples of processing type errors include, without limitation: an answer which is very far from the correct answer, such as 1+1=1500; an answer which is not relevant to the procedure required in the question, such as performing addition instead of subtraction; and/or an answer which has the correct parts, but does not have any relationship between the different parts of the answer.

In another embodiment, as will be described below, the response time to a respective exercise, i.e. the amount of time which from the output of the respective exercise to receipt of the user response, is measured and a processing error type is determined responsive to the measured response time in a plurality of exercises. For example, if the response time is faster than a predetermined processing speed threshold value, and in the event that this response time is generally fixed for a plurality of different exercises, exhibiting different level of difficulties, it is determined that these answers are processing type errors. In one embodiment, the response time in a respective exercise is measured only if the answer is incorrect. In another embodiment, the response time in a respective exercise is measured for all answers.

Profile model database 150 has stored therein a respective model for each of a plurality of field profiles. Particularly, as will be described below, processor 30 determines, for each of the plurality of error types, whether the user overall exhibits the respective error type. Each model represents a respective combination of the plurality of error types. Table 1 shows one embodiment of the plurality of field profiles and their respective models:

TABLE 1

| Profile: | A | B | C | D | E | F | G | T |
|---|---|---|---|---|---|---|---|---|
| Input | + | − | − | + | + | − | − | + |
| Output | + | + | − | − | − | − | + | + |
| Process | − | − | − | − | + | + | + | + |

As shown in Table 1, each field profile (A, B, C, D, E, F, G and T) has a unique combination of overall user error types, where a minus sign indicates that the user does not exhibit such an error type and a plus sign indicates that the user doe exhibit such an error type. Thus, each model comprises the respective combination of user error types, as shown. The term “field profile”, as used herein, is meant as a profile associated with a respective cognitive field. Particularly, a user may have different profiles for different cognitive fields.

Improvement exercise database 160 comprises data regarding a plurality of improvement exercises. A respective set of the plurality of improvement exercises is associated with each of the plurality of field profiles. In one embodiment, the sets overlap, i.e. one or more exercises are associated with more than one field profile. The data regarding the improvement exercises comprises sequence data, i.e. in what sequence each set of improvement exercises should be output in. The improvement exercises are aimed at improving the cognitive abilities of the user, in accordance with their respective field profile. Thus, the exercises which aim to improve the cognitive abilities of the user are specifically aimed at the user's specific problems, which are defined by the user's field profile, thereby improving the efficacy of the exercises.

Specifically, for a user with field profile A, a set of improvement exercises primarily focused on process difficulties are utilized. For a user with field profile B, sets of improvement exercises primarily focused on input and process difficulties are utilized. For a user with field profile C, sets of improvement exercises focused on all three type of difficulties are utilized. For a user with field profile D, sets of improvement exercises primarily focused on output and process difficulties are utilized. For a user with field profile E, a set of improvement exercises primarily focused on output difficulties are utilized. For a user with field profile F, sets of improvement exercises primarily focused on input and output difficulties are utilized. For a user with field profile G, a set of improvement exercises primarily focused on input difficulties are utilized. For a user with field profile T, the respective set of improvement exercises are focused across a range of difficulties, i.e. the selected set is not focused on any specific error type.

In one embodiment, different sub-sets of improvement exercises are provided, each sub-set associated with a common problem, such that each improvement exercise comprises a different way of displaying the common problem. For example, the following improvement exercises are all associated with the mathematical equation: 4+4=8.

For improvement exercises associated with an input error type, several improvement exercises include: instructing the user to click with a pointer on the first numeral in the problem and then on the second numeral; instructing the user to separately click on each numeral and outputting an audio signal of the numeral which is selected; instructing the user to separately click on each numeral and adjusting the font, or font size, of the selected numeral; and displaying the problem 4+4=? for a predetermined amount of time and then hiding the problem and instructing the user to recall the question by selecting one of a plurality of options.

For improvement exercises associated with a processing error type, several improvement exercises include the following. 1. Two groups of fruit, each group with four fruits, are displayed, with a plus sign between them. Additionally, a basket is displayed and the user is instructed to select one of a plurality of options for what to do to get the answer. A correct answer would be putting both fruits in the basket. 2. Several options for answers which aren't close to the correct answer, such as 500 and 1000, are displayed and several options which are close, such as 3 and 5, are displayed. The user is then instructed to select the answers which could be correct. 3. A plurality of possible answers are displayed, separately, such as 6, 7, 8 and 10. For each of the displayed answers, the user is instructed to select from a plurality of options why the answer can, or can't be correct, e.g. the answer is too high or too low.

For improvement exercises associated with an output error type, several improvement exercises include the following. 1. The entire equation is displayed for a predetermined amount of time. Then, the question is hidden and only the solution is displayed. The user is then instructed to type in the correct question from a plurality of options and further select a correct explanation from a plurality of options why that has to be the correct question. 2. After the user has answered the question, the user is instructed to type the question. 3. The user is instructed to pass the cursor over each word in the question, otherwise the exercise cannot be completed.

In one embodiment, each of the improvement exercises has associated therewith, for each of the plurality of predetermined error types, respective parameter limits indicative of the respective error type, as described above in relation to the diagnostic exercises.

Thus, for each cognitive field, improvement exercise database has stored therein a respective set of improvement exercises for each of the plurality of profiles, each of the improvement exercises associated with a respective difficulty level.

In one embodiment, the diagnostic and/or improvement exercises stored in memory 40 are updated periodically from optional server 130.

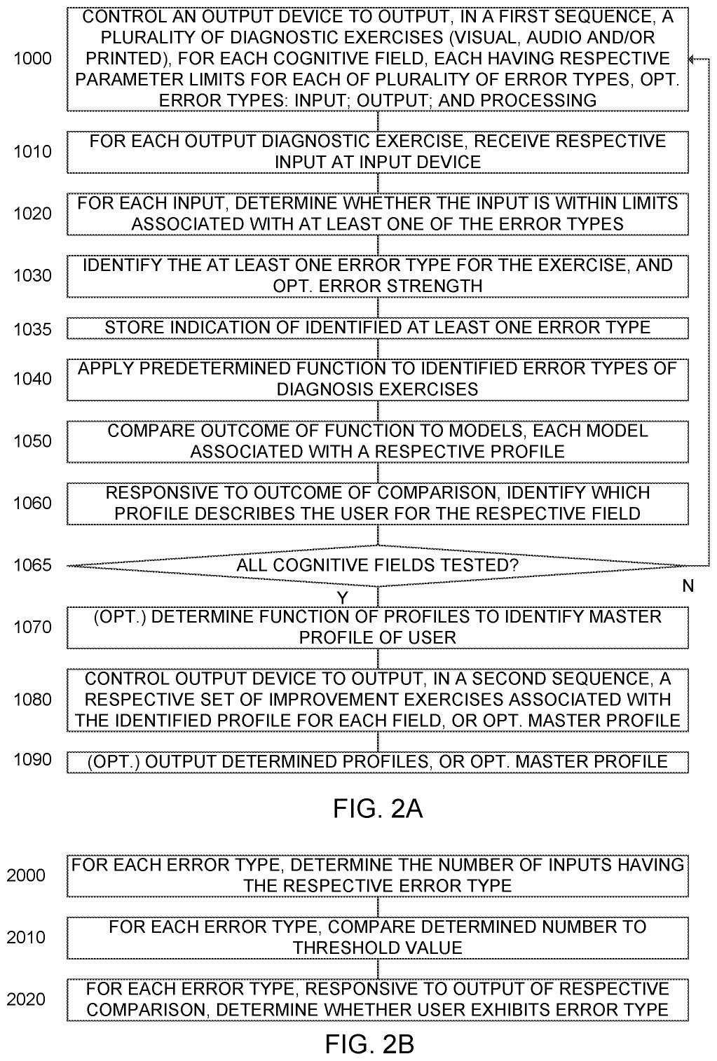

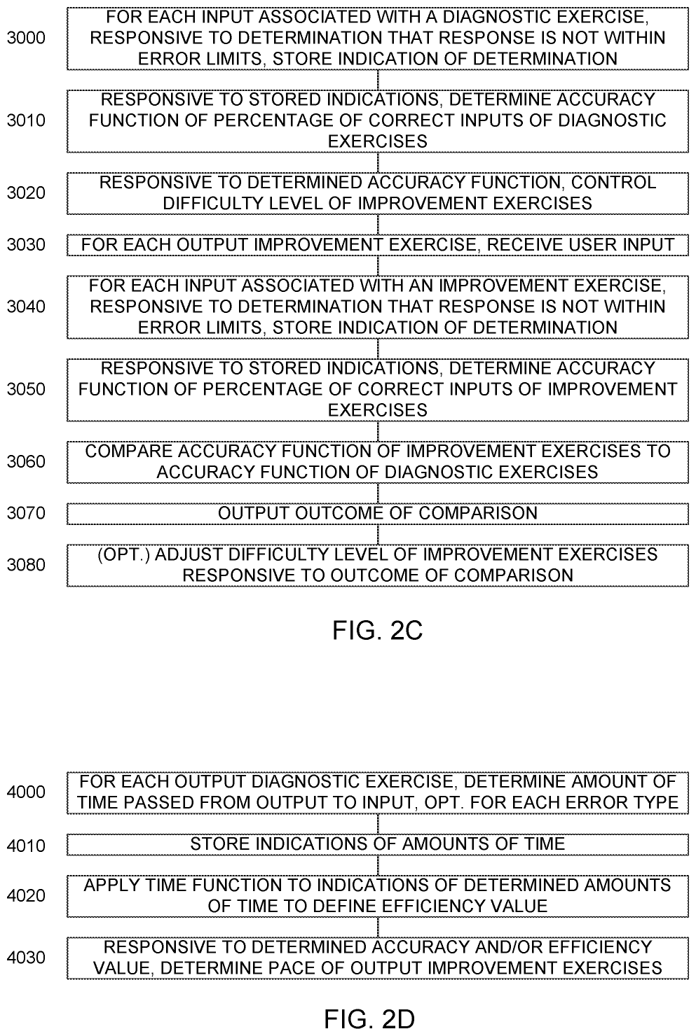

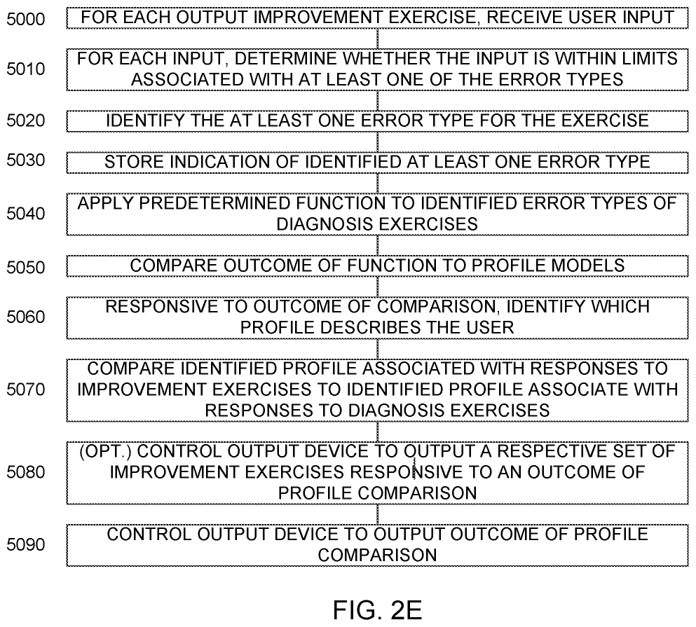

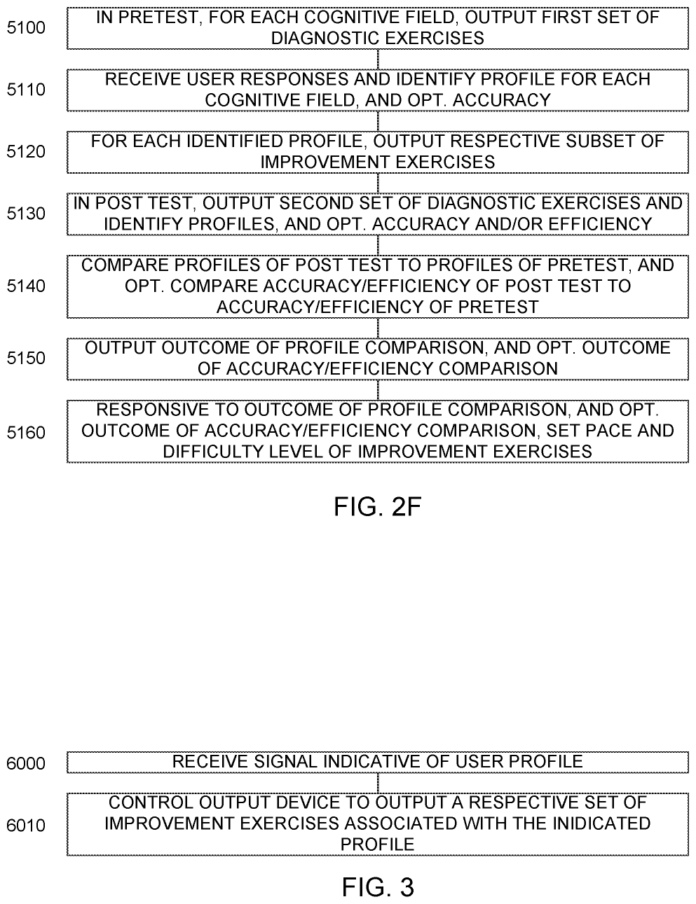

FIG. 2A illustrates a high level flow chart of a first method of operation of profile oriented cognitive improvement system 10, FIGS. 1A-2A being described together. In stage 1000, exercise selection module 90 of processor 30 selects from diagnostic exercise database 140 a group of diagnostic exercises and begins a diagnostic test. As described above, each group of diagnostic exercises is associated with a respective cognitive field. Stage 1000, and stages 1010-1060 described below, are performed separately for each cognitive field, i.e. for each group of diagnostic exercises. In one embodiment, not all of the cognitive fields are tested and stages 1000-1060 are performed for a set of the plurality of cognitive fields, the set of cognitive fields optionally Output module 100 controls output device 50 to output the selected group of diagnostic exercises in a predetermined sequence.

Output module 100 controls output device 50 to output the selected group of diagnostic exercises one by one, each output diagnostic exercise providing a user, supported by user support 70, a question to answer or task to complete. Specifically, each output diagnostic exercise prompts the user to input a response at input device 60. After an input is received at input device 60, output module 100 controls output device 50 to output the next diagnostic exercise of the group. This continues until the group of diagnostic exercises, selected by exercise selection module 90, is completed. The respective diagnostic exercise being output can be output as: a visual exercise, i.e. the question and optionally possible answers are displayed on the screen of output device 50; and/or an audio exercise, i.e. the question and optionally possible answers are output by a speaker system of output device. As described above, each diagnostic exercise has associated therewith respective parameter limits for each of a plurality of error types. As further described above, the error types include: input error type; output error type; and processing error type.

In stage 1010, for each of the output diagnostic exercises of stage 1000, input module 110 receives a user input through input device 60. As described above, the user input can optionally be received from a mouse, a keyboard and/or a touch screen. In stage 1020, for each received input of stage 1010, analysis module 120 determines whether the received input is within the predetermined parameter limits of the respective diagnostic exercise associated with at least one of the plurality of error types, i.e. analysis module 120 determines whether there was an error of at least one of the error types in the user response. Particularly, a user response may contain more than one type of error. As described above, in one embodiment, certain errors are detected only after analyzing the responses to a number of output diagnostic exercises. In stage 1030, responsive to the determination of stage 1020, analysis module 120 identifies the at least one error type within the respective user response and in stage 1035 stores an indication of the identified error type, or error types, in memory 40. In one embodiment, analysis module 120 further determines the strength of the error, i.e. how wrong the user response was. For example, in a diagnostic exercise with a plurality of selectable answers, some answers can indicate a larger error than other answers. Thus, the answer selected is compared to the predetermined parameter limits not only to determine whether there is an error or not, but what the strength of the error is. In one embodiment, each answer is assigned a respective error strength corresponding to the difference between the respective answer and the correct answer. In an embodiment where the error strength of the user response is determined, the error strength is stored in memory 40.

In stage 1040, after outputting the selected group of diagnostic exercises of stage 1000, analysis module 120 applies a predetermined error function to the stored indications of the identified error types of stage 1035. In one embodiment, the data associated with the diagnostic exercises, stored on diagnostic exercise database 140, further comprises a statistical model indicating an acceptable number of errors, and their respective types for the selected group of diagnostic exercises. In one further embodiment, the acceptable number of errors depends on the respective exercises, i.e. for certain diagnostic exercises a smaller number of errors of a particular type is considered acceptable and for other diagnostic exercises a larger number of errors of a particular type is considered acceptable. The predetermined error function is thus related to the statistical model. For example, in one embodiment, the predetermined error function determines whether the respective number of errors of a specific type exceeds the maximum number of allowed errors of the respective statistical model. In the event that the respective number of errors exceeds the maximum number of allowed errors, the predetermined error function outputs that the user exhibits the respective error type, i.e. the user has a cognitive problem of that respective type. It is noted that the user can exhibit more than one error type, as described above in relation to Table 1.

FIG. 2B illustrates a high level flow chart of a more detailed embodiment of the process of stage 1040. Particularly, in stage 2000, for each of the plurality of error types, the number of diagnostic exercises whose respective inputs have the respective error type is determined by analysis module 120, as described above. In stage 2010, for each of the plurality of error types, analysis module compares the determined number of stage 2000 to a respective predetermined error threshold value. As described above, in one embodiment, the predetermined error threshold value is based on a statistical model. In stage 2020, for each of the plurality of error types, responsive to the outcome of the comparison of stage 2010, analysis module 120 determines whether the user exhibits the respective error type. Particularly, as described above, in the event that the number of identified errors of the respective type exceeds the respective error threshold value, analysis module 120 determines that the user exhibits the respective error type. The models stored in profile model database 150 comprise a respective combination of user error types, as described above in relation to Table 1.

Returning to FIG. 2A, in stage 1050, analysis module 120 compares an outcome of the function of stage 1040 with the plurality of models stored in profile model database 150. As described above, each model is associated with a respective cognitive field profile. In stage 1060, responsive to the outcome of the comparison of stage 1050, analysis module 120 determines which field profile describes the user. Particularly, in one embodiment, where the function of stage 1040 outputs the error types associated with the user, analysis module 120 compares the identified one or more error types to the models to determine which model meets the identified error types, as described above in relation to the example of Table 1.

In one embodiment, where the error strengths of the user responses are determined, analysis module 120 further determines a strength value of the identified field profile. Particularly, a predetermined function of the determined error strengths of the user responses is determined, the function of the determined error strengths defining the strength value of the identified field profile.

As described above, stages 1000-1060 are performed separately for each of the selected cognitive fields, i.e. for each group, or selected subgroup, of diagnostic exercises. Thus, a respective field profile is determined for each cognitive field. In stage 1065 it is determined whether all of the cognitive fields have been tested. As described above, in one embodiment only a respective set of cognitive fields are tested. In such an embodiment, it is determined whether all of the cognitive fields of the respective set have been tested. If in stage 1065 all of the cognitive field have not yet been tested, the method proceeds to stage 1000 on the next cognitive field. In the event that in stage 1065 it is determined that all of the cognitive fields have been tested, in optional stage 1070, a predetermined function is determined for the plurality of field profiles to identify whether the user has a common profile, i.e. a field profile which represents the user in all, or most cognitive fields. Particularly, in one embodiment, the number of cognitive fields which have the same field profile for the user is determined. In the event that the number which have the same field profile is greater than a predetermined threshold, that field profile is considered the common profile of the user. In the event that the number is not greater than the predetermined threshold, it is determined that the user does not have a common profile.

In stage 1080, responsive to the identified field profiles of stage 1060 and the common profile determination of stage 1070, exercise selection module 90 selects from improvement exercise database 160 a respective portion of the respective sets of improvement exercises which is associated with the identified field profiles. Particularly, as described above, each field profile has associated therewith a set of improvement exercises. In one embodiment, in the event that a common profile was identified in optional stage 1070, a set of improvement exercises associated with the common profile is selected. In the event that it was determined that there is no common profile, sets of improvement exercises for each of the cognitive fields identified in stage 1060 are selected.

In one embodiment, as will be described below in relation to FIG. 2C, not all of the exercises of the associated set are selected, the particular improvement exercises of the respective set is selected responsive to the percentage of correct answers received from the diagnostic exercises. Output module 100 controls output device 50 to output the selected improvement exercises in a predetermined sequence. Each of the output improvement exercises prompts the user to input a response at input device 60, as described above in relation to the output diagnostic exercises. In one embodiment, the selected improvement exercises are printed out by output device 50 so the user can perform the improvement exercises without the use of a computer, which is advantageous for some people who have difficulty performing the exercises electronically.

In optional stage 1090, output module 100 controls output device 50 to output an indication of the identified field profiles of stage 1060 and/or the identified common profile of optional stage 1070, if there is a common profile.

FIG. 2C illustrates a high level flow chart of an embodiment of controlling which improvement exercises are selected in stage 1080. In stage 3000, for each received input of stage 1010, associated with a respective output diagnostic exercise, analysis module 120 determines whether the response falls within the predetermined parameter limits of any of the error types, as described above in relation to stage 1020. Responsive to a determination that the response does not fall within the predetermined parameter limits of any of the error types, i.e. the answer is correct, or correct enough to not be considered in this case an error, analysis module 120 stores an indication of such a determination in memory 40.

In stage 3010, responsive to the stored indications of stage 3000, an accuracy value of a percentage of correct inputs of the diagnostic exercises is determined, i.e. those inputs which did not fall within any of the predetermined parameter limits for the error types. In one embodiment, the accuracy value is merely the percentage value itself. In another embodiment, the accuracy value is the total number of correct inputs, thus reflecting the percentage of correct inputs. Such an embodiment is optionally performed when the number of diagnosis and improvement exercises provided to the user are the same.

In one embodiment, a respective accuracy value is determined for each cognitive field. In such an embodiment, a predetermined function is determined for the plurality of accuracy values to identify whether the user has a common accuracy value, i.e. an accuracy value which represents the user in all, or most cognitive fields. In one embodiment, it is determined whether the accuracy values are within a predetermined range from each other. In the event that the accuracy values are within a predetermined range from each other, a predetermined function of the accuracy values is defined as a common accuracy value. In one non-limiting embodiment, the predetermined function is an average of the accuracy values. In the event that the accuracy values are not within the predetermined range from each other, it is determined that there is no common accuracy value.

In stage 3020, for each cognitive field, responsive to the determined accuracy value of stage 3010, exercise selection module 90 controls which particular exercises of the respective set of selected improvement exercises of stage 1070 are selected. Particularly, each set of improvement exercises comprises exercises of varying difficulty levels and the desired difficulty level is selected responsive to the determined accuracy. In one embodiment, for a greater accuracy, improvement exercises of a greater difficulty level are selected. For a lower accuracy, improvement exercises of a lower difficulty level are selected.

In the event that in stage 3010 a common accuracy value is defined, the difficulty level is selected responsive to the common accuracy value in all of the cognitive fields.

In stage 3030, for each output improvement exercise of stage 1070, input module 110 receives an input entered at input device 60. In stage 3040, for each received input of stage 3030, associated with a respective output improvement exercise, analysis module 120 determines whether the response falls within the predetermined parameter limits which define any of the error types, as described above in relation to stage 3000 regarding responses to the diagnostic exercises. Responsive to a determination that the response does not fall within the predetermined parameter limits of any of the error types, i.e. the answer is correct, or correct enough to not be considered in this case an error, analysis module 120 stores an indication of such a determination in memory 40.

In stage 3050, responsive to the stored indications of stage 3040, analysis module determines an accuracy value of a percentage of correct inputs of the improvement exercises, i.e. those inputs which did not fall within any of the predetermined parameter limits for the error types, as described above in relation to stage 3010.

In stage 3060, analysis module 120 compares the determined accuracy value of stage 3050 to the determined accuracy value of stage 3010, i.e. the determined accuracy of the responses to the improvement exercises is compared to the determined accuracy of the responses to the diagnostic exercises. In stage 3070, analysis module 120 outputs the outcome of the comparison of stage 3060. In one embodiment, the outcome of the comparison is output to output module 100 and output module 100 controls output device 50 to output the outcome of the comparison, i.e. to output to the user the change in accuracy of the user's responses.

In optional stage 3080, in an embodiment where additional improvement exercises are output to the user, exercise selection module 90 controls a difficulty level of which improvement exercises are selected, in accordance with their respective difficulty levels, responsive to the outcome of the comparison of stage 3070. Particularly, in one embodiment, if there is an improvement in the accuracy of the responses, improvement exercises with a greater difficulty level are selected.

FIG. 2D illustrates a high level flow chart of an embodiment of determining a pace of the output improvement exercises of stage 1080. In stage 4000, for each output diagnostic exercise of stage 1000, analysis module 120 determines the amount of time passed from the time that the exercise was output in stage 1000 until the respective input was received in stage 1010.

In stage 4010, analysis module 120 stores an indication of each of the determined amounts of time of stage 4000 in memory 40. In stage 4020, analysis module 120 determines a predetermined time function of the stored indications of the determined amounts of time of stage 4000, the outcome of the predetermined time function defining an efficiency value. In one embodiment, the predetermined time function comprises an average. In another embodiment, different improvement exercises are assigned different weights.

In one embodiment, the amount of time passed is determined separately for each error type. Particularly, for all of the user responses determined to be of a respective error type, a predetermined function of the amount of time passed for each respective exercise is determined. For example, if the user responses for exercises 1, 2, 3, 5 and 7 are determined to be of an input error type, an average of the times for each of exercises 1, 2, 3, 5 and 7 is determined. The determined time average defines the efficiency value for the input error type of user responses.

In one embodiment, a respective efficiency value is determined for each cognitive field. In such an embodiment, a predetermined function is determined for the plurality of efficiency values to identify whether the user has a common efficiency value, i.e. an efficiency value which represents the user in all, or most cognitive fields. In one embodiment, it is determined whether the efficiency values are within a predetermined range from each other. In the event that the efficiency values are within a predetermined range from each other, a predetermined function of the efficiency values is defined as a common efficiency value. In one non-limiting embodiment, the predetermined function is an average of the efficiency values. In the event that the efficiency values are not within the predetermined range from each other, it is determined that there is no common efficiency value.

In stage 4030, for each cognitive field, responsive to the respective determined efficiency value of stage 4020, and/or responsive to the determined accuracy value of stage 3050, output module 100 determines the pace of output of the improvement exercises of stage 1070. Particularly, in one embodiment, responsive to the efficiency value indicating that the user responds quickly, and/or responsive to the determined accuracy value indicating that the user answers accurately, analysis module 120 determines an efficiency score for the user. Responsive to a high determined efficiency score, output module 100 increases the pace of outputting the improvement exercises by output device 50. In one embodiment, an increase of pace comprises displaying the improvement exercise for less time, thereby allowing the user to perform more exercises in a predetermined amount of time. A decrease of pace comprises displaying the improvement exercise for more time, thereby allowing the user to perform fewer exercises in the predetermined amount of time. In another embodiment, an increase of pace comprises reducing the number of output improvement exercises before the difficulty level of the improvement exercises is increased, and reducing the pace comprises increasing the number of output improvement exercises before the difficulty level of the improvement exercises is increased. In the event that a common efficiency value is defined, the pace is determined responsive to the common efficiency value in all of the cognitive fields.

In one embodiment, the pace is determined responsive to a predetermined function of the accuracy and efficiency value. In one further embodiment, the pace is determined responsive to a multiplication of the efficiency value and the accuracy value.

In the embodiment where the efficiency value is determined for each error type, the pace is adjusted separately for the improvement exercises associated with each respective error type in accordance with the respective efficiency value.

FIG. 2E illustrates a high level flow chart of a method of analyzing a profile change of a user. In stage 5000, for each of the output improvement exercises of stage 1080, input module 110 receives a respective user input from input device 60. In stage 5010, for each received input of stage 5000, analysis module 120 determines whether the received input is within the predetermined parameter limits of the respective improvement exercise associated with at least one of the plurality of error types, i.e. analysis module 120 determines whether there was an error of at least one of the error types in the user response. In stage 5020, responsive to the determination of stage 5010, analysis module 120 identifies the at least one error type within the respective user response and in stage 5030 stores an indication of the identified error type, or error types, in memory 40.

In stage 5040, after outputting the selected portion of the respective set of improvement exercises of stage 1070, analysis module 120 applies a respective predetermined error function to the stored indications of the identified error types of stage 5030, as described above in relation to stage 1040 regarding the identified errors in the responses to the diagnostic exercises.

In stage 5050, analysis module 120 compares an outcome of the error function of stage 5040 with the plurality of models stored in profile model database 150. As described above, each model is associated with a respective cognitive field profile. In stage 5060, responsive to the outcome of the comparison of stage 5050, analysis module 120 determines which field profile describes the user, as described above in relation to stage 1060 regarding the received responses to the diagnostic exercises.

In stage 5070, analysis module 120 compares the determined field profile of stage 5060, associated with the output improvement exercises, with the determined field profile of stage 1060, associated with the output diagnostic exercises.

In optional stage 5080, responsive to the outcome of the comparison of stage 5070, exercise selection module 90 selects from improvement exercise database 160 a portion of a respective set of improvement exercises and output module 100 controls output device 50 to output the selected improvement exercises. Particularly, in one embodiment, in the event that the outcome of the comparison of stage 5060 indicates that the field profile of the user hasn't changed, a larger number of improvement exercises than what was output in stage 1080 are output. In the event that the outcome of the comparison of stage 5060 indicates that the field profile of the user has changed, a reduced number of improvement exercises than what was output in stage 1080 are output, and additional improvement exercises responsive to the changed user field profile are output. In stage 5090, analysis module 120 outputs the outcome of the comparison of stage 5070, i.e. the change in field profile of the user. In one embodiment, the outcome of the comparison is output to output module 100 and output module 100 control output device 50 to output the outcome of the comparison, i.e. to output to the user the updated user cognitive field profile. Outputting the change in field profile indicates to the user their learning ability.

FIG. 2F illustrates a high level flow chart of a method of a profile diagnosis, utilizing a pretest and a post test. In stage 5100, exercise selection module 90 of processor 30 selects from diagnostic exercise database 140 a first set of groups of diagnostic exercises, each group associated with a respective cognitive field, as described above in relation to stage 1000. The selected diagnostic exercises are output at output device 50, as described above. In stage 5110, the respective user responses to the output diagnostic exercises are received and a field profile of the user, for each cognitive field, is identified, as described above. Additionally, in one embodiment, the accuracy of the user responses is determined, as described above in relation to stage 3050. Stages 5100-5110 thus constitute a pretest. In stage 5120, for each of the identified field profiles of stage 5110, or for an optionally identified common profile, a respective subset of improvement exercises are output at output device 50, as described above in relation to stage 1080. Stage 5120 is defined as the intervention phase of the diagnostic routine. In one embodiment, the subset of improvement exercises comprises only a portion of the respective set of improvement exercises described above.

In stage 5130, in a post test phase, stages 5100-5110 are again performed, preferably with diagnostic exercises different than those selected in stages 5100-5110. Particularly, the field profile of the user for each cognitive field is identified. Additionally, in one embodiment, the accuracy value of the user responses is also determined. In another embodiment, the efficiency value of the user responsive is also determined. In stage 5140, the identified field profiles of stage 5130 are compared to the identified field profiles of stage 5110. Additionally, in one embodiment, the determined accuracy value of stage 5130 is compared to the determined accuracy value of stage 5110. Additionally, in one embodiment, the determined efficiency value of stage 5130 is compared to the determined efficiency value of stage 5110. Thus, the comparisons of stage 5140 determined the cognitive improvement potential of the user. Particularly, the larger the difference between the values in the pre-test and the post-test, the greater the potential of the user is.

In stage 5150, the outcome of the profile comparison of stage 5140 is output at output device 50. In one embodiment, the outcome of the accuracy and/or efficiency comparison of stage 5140 is output. In stage 5160, responsive to the outcome of the profile comparison of stage 5140, and optionally responsive to the outcome of the accuracy and/or efficiency comparison of stage 5140, the difficulty level and pace of the improvement exercises of stage 1080 is set, as described above in relation to stages 3080 and 4030. In one embodiment, for a greater identified potential, improvement exercises of a greater difficulty level are selected and presented. For a lower identified potential, improvement exercises of a lower difficulty level are selected and presented.

Thus, in one embodiment, improvement exercises are selected responsive to: the identified profile; the identified accuracy value; and the identified efficiency value.

FIG. 3 illustrates a high level flow chart of a second method of operation of profile oriented cognitive improvement system 10. In stage 6000, a signal is received, the received signal indicative of a respective one of a plurality of cognitive field profiles. In one embodiment, the received signal comprises an input signal received from input device 60. In another embodiment, the received signal comprises an input signal received from optional server 130 responsive to a user input received through input device 60. In stage 6010, responsive to the received indication of the user field profile of stage 6000, exercise selection module 90 selects from improvement exercise database 160 a respective portion of the respective set of improvement exercises which is associated with the indicated field profile, as described above in relation to stage 1080. As further described above, output module 100 controls output device 50 to output the selected improvement exercises.

Thus, the above embodiments provide a diagnostic method and associated improvement therapy controlled throughout the process to ensure ongoing consonance with an ongoing diagnostic model. It is noted that the inventors have discovered that the above methods not only improve cognitive functions, but also cause physical improvements in the brains of patients with cognitive deficiencies.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

Unless otherwise defined, all technical and scientific terms used herein have the same meanings as are commonly understood by one of ordinary skill in the art to which this invention belongs. Although methods similar or equivalent to those described herein can be used in the practice or testing of the present invention, suitable methods are described herein.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

The terms “include”, “comprise” and “have” and their conjugates as used herein mean “including but not necessarily limited to”.

It will be appreciated by persons skilled in the art that the present invention is not limited to what has been particularly shown and described hereinabove. Rather the scope of the present invention is defined by the appended claims and includes both combinations and sub-combinations of the various features described hereinabove as well as variations and modifications thereof, which would occur to persons skilled in the art upon reading the foregoing description.

1. A profile oriented cognitive improvement system comprising:
a processor;
an output device arranged to provide a user with visual, audio or printed output responsive to said processor;
an input device arranged to receive a user input; and
a memory having stored thereon:
data regarding a plurality of diagnostic exercises, each of said plurality of diagnostic exercises having associated therewith, for each of a plurality of predetermined error types, respective parameter limits indicative of said respective error type;
data regarding a plurality of models, each of said plurality of models associated with a respective one of a plurality of field profiles, each of said plurality of field profiles associated with a respective one of said plurality of predetermined error types; and
data regarding a plurality of improvement exercises, each of a plurality of sets of said plurality of improvement exercises associated with a respective one of said plurality of field profiles;
wherein said processor is arranged to:
control said output device to output, in a first predetermined sequence, the plurality of diagnostic exercises, each of the plurality of diagnostic exercises prompting a user to input a response at said input device;
for each of said output diagnostic exercises, receive a respective input at said input device;
for each of said received inputs, determine whether said respective received input is within said predetermined parameter limits of said respective diagnostic exercise associated with at least one of said plurality of predetermined error types;
for each of said received inputs, responsive to said determination that said respective received input is within said predetermined parameter limits associated with said at least one of said plurality of predetermined error types, identify said at least one of said plurality of predetermined error types;
for each of said received inputs, responsive to said identification, store an indication of said identified at least one of said plurality of predetermined error types on said memory;
apply a predetermined first function to said indications of said identified error types for said received inputs;
compare an output of said applied predetermined first function to each of said plurality of models stored on said memory;
responsive to an outcome of said comparison, identify which of said plurality of field profiles describes the user;
control said output device to output, in a second predetermined sequence, a respective portion of said respective set of said plurality of improvement exercises associated with said identified field profile, each of said plurality of improvement exercises prompting a user to input a response at said input device.

2. The system of claim 1, wherein said plurality of predetermined error types comprises: input error type; output error type; and processing error type.

3. The system of claim 1, wherein said processor is further arranged to:
for each of said predetermined error types, determine the number of said received inputs having said respective error type;
for each of said predetermined error types, compare said determined number to a respective predetermined error threshold value; and
for each of said predetermined error types, responsive to an output of said comparison to said respective predetermined error threshold value, determine whether the user exhibits said respective error type,
wherein said applied predetermined first function comprises said determination of said error type exhibition of the user, and
wherein each of said plurality of models comprises a respective combination of said predetermined error types.

4. The system of claim 1, wherein said processor is further arranged to:
for each of said received inputs, associated with a respective one of said output diagnostic exercises, and responsive to a determination that said respective received input is not within said respective predetermined parameter limits associated with any of said plurality of predetermined error types, store on said memory an indication of said determination;
responsive to said stored indications of said determinations, determine a predetermined accuracy value of a percentage of correct inputs associated with said output diagnostic exercises; and
responsive to said determined accuracy value, determines a level of difficulty of said portion of said output improvement exercises.

5. The system of claim 4, wherein said data regarding each of said plurality of improvement exercises comprises, for each of said plurality of predetermined error types, respective parameter limits indicative of said respective error type, and
wherein said processor is further arranged to:
for each of said output improvement exercises, receive a respective input at said input device;
for each of said received inputs, associated with a respective output improvement exercise, and responsive to a determination that said respective received input is not within said respective predetermined parameter limits associated with any of said plurality of predetermined error types, store on said memory an indication of said determination;
responsive to said stored indications of said determinations, associated with said output improvement exercises, determine said accuracy value of a percentage of correct inputs associated with said output improvement exercises;
compare an outcome of said accuracy value of said percentage of correct inputs associated with said output improvement exercises with said outcome of said accuracy value of said percentage of correct inputs associated with said output diagnostic exercises; and
output an outcome of said accuracy value outcome comparison.

6. The system of claim 4, wherein, responsive to said determined accuracy value of said percentage of correct input associated with said output diagnostic exercises, said processor is further arranged to control a pace of said output improvement exercises.

7. The system of claim 1, wherein said processor is further arranged to:
for each of said output diagnostic exercises, determine the amount of time which passed from said respective output to said respective received input;
determine a predetermined time function to said determined amounts of time; and
control a pace of said output improvement exercises responsive to said determined time function.

8. The system of claim 1, wherein said data regarding each of said plurality of improvement exercises comprises, for each of said plurality of predetermined error types, respective parameter limits indicative of said respective error type, and
wherein said processor is further arranged to:
for each of said output improvement exercises, receive a respective input at said input device;
for each of said received inputs, associated with a respective improvement exercise, determine whether said respective received input is within said predetermined parameter limits of said respective improvement exercise associated with at least one of said plurality of predetermined error types;
for each of said received inputs, associated with a respective improvement exercise, and responsive to said determination that said respective received input is within said predetermined parameter limits associated with said at least one of said plurality of predetermined error types, identify said at least one of said plurality of predetermined error types;
for each of said received inputs, associated with a respective improvement exercise, responsive to said error type identification, store an indication of said identified at least one of said plurality of predetermined error types on said memory;
apply said predetermined first function to said indications of said identified error types for said received inputs associated with said respective output improvement exercises;
compare an output of said applied predetermined first function, associated with said respective output improvement exercises, to each of said plurality of models stored on said memory;
responsive to an outcome of said comparison, associated with said respective output improvement exercises, identify which of said plurality of field profiles describes the user;
compare said identified field profile of the user, associated with said output improvement exercises, with said identified field profile of the user associated with said output diagnostic exercises; and
control said output device to output a respective portion of a respective set of said plurality of improvement exercises, said respective portion of said respective set responsive to an outcome of said comparison of said identified field profiles.

9. The system of claim 8, wherein said processor is further arranged to output an outcome of said comparison of said identified field profiles.

10. The system of claim 1, wherein said plurality of diagnostic exercises comprises a plurality of groups of diagnostic exercises, each of said plurality of groups associated with a respective one of a plurality of cognitive fields, said field profile identified separately for each of the plurality of cognitive fields, and
wherein said plurality of sets of improvement exercises comprises a plurality of groups of sets of improvement exercises, each of said plurality of groups associated with a respective one of the plurality of cognitive fields, said output of said improvement exercises being for each of the plurality of cognitive fields.

11. The system of claim 1, wherein said processor is further arranged to identify, responsive to said identified field profile of each of the plurality of cognitive fields, a common profile which described the user, said identified common profile responsive to a predetermined common profile function of said identified field profiles of the plurality of cognitive fields.

12. A profile oriented cognitive improvement method, the method comprising:
storing on a memory data regarding a plurality of diagnostic exercises, each of said plurality of diagnostic exercises having associated therewith, for each of a plurality of predetermined error types, respective parameter limits indicative of said respective error type;
storing on the memory data regarding a plurality of models, each of said plurality of models associated with a respective one of a plurality of field profiles, each of said plurality of field profiles associated with a respective one of said plurality of predetermined error types;
storing on the memory data regarding a plurality of improvement exercises, each of a plurality of sets of said plurality of improvement exercises associated with a respective one of said plurality of field profiles;
controlling an output device to output, in a first predetermined sequence, the plurality of diagnostic exercises, each of the plurality of diagnostic exercises prompting a user to input a response at an input device;
for each of said output diagnostic exercises, receiving a respective input at said input device;
for each of said received inputs, determining whether said respective received input is within said predetermined parameter limits of said respective diagnostic exercise associated with at least one of said plurality of predetermined error types;
for each of said received inputs, responsive to said determination that said respective received input is within said predetermined parameter limits associated with said at least one of said plurality of predetermined error types, identifying said at least one of said plurality of predetermined error types;
for each of said received inputs, responsive to said identification, storing an indication of said identified at least one of said plurality of predetermined error types on said memory;
applying a predetermined first function to said indications of said identified error types for said received inputs;
comparing an output of said applied predetermined first function to each of said plurality of models stored on said memory;
responsive to an outcome of said comparison, identifying which of said plurality of field profiles describes the user;
controlling said output device to output, in a second predetermined sequence, a respective portion of said respective set of said plurality of improvement exercises associated with said identified field profile, each of said plurality of improvement exercises prompting a user to input a response at said input device.

13. The method of claim 12, wherein said plurality of predetermined error types comprises: input error type; output error type; and processing error type.

14. The method of claim 12, further comprising:
for each of said predetermined error types, determining the number of said received inputs having said respective error type;
for each of said predetermined error types, comparing said determined number to a respective predetermined error threshold value; and
for each of said predetermined error types, responsive to an output of said comparison to said respective predetermined error threshold value, determining whether the user exhibits said respective error type,
wherein said applied predetermined first function comprises said determination of said error type exhibition of the user, and
wherein each of said plurality of models comprises a respective combination of said predetermined error types.

15. The method of claim 12, further comprising:
for each of said received inputs, associated with a respective one of said output diagnostic exercises, and responsive to a determination that said respective received input is not within said respective predetermined parameter limits associated with any of said plurality of predetermined error types, storing on said memory an indication of said determination;
responsive to said stored indications of said determinations, determining a predetermined accuracy value of a percentage of correct inputs associated with said output diagnostic exercises; and
responsive to said determined accuracy value, determining a level of difficulty of said portion of said output improvement exercises.

16. The method of claim 15, wherein said data regarding each of said plurality of improvement exercises comprises, for each of said plurality of predetermined error types, respective parameter limits indicative of said respective error type, and
wherein the method further comprises:
for each of said output improvement exercises, receiving a respective input at said input device;
for each of said received inputs, associated with a respective output improvement exercise, and responsive to a determination that said respective received input is not within said respective predetermined parameter limits associated with any of said plurality of predetermined error types, storing on said memory an indication of said determination;
responsive to said stored indications of said determinations, associated with said output improvement exercises, determining said accuracy value of a percentage of correct inputs associated with said output improvement exercises;
comparing an outcome of said accuracy value of said percentage of correct inputs associated with said output improvement exercises with said outcome of said accuracy value of said percentage of correct inputs associated with said output diagnostic exercises; and
outputting an outcome of said accuracy value outcome comparison.

17. The method of claim 15, wherein, responsive to said determined accuracy value of said percentage of correct input associated with said output diagnostic exercises, the method further comprises controlling a pace of said output improvement exercises.

18. The method of claim 12, further comprising:
for each of said output diagnostic exercises, determining the amount of time which passed from said respective output to said respective received input;
determining a predetermined time function to said determined amounts of time; and
controlling a pace of said output improvement exercises responsive to said determined time function.

19. The method of claim 12, wherein said data regarding each of said plurality of improvement exercises comprises, for each of said plurality of predetermined error types, respective parameter limits indicative of said respective error type, and
wherein the method further comprises:
for each of said output improvement exercises, receiving a respective input at said input device;
for each of said received inputs, associated with a respective improvement exercise, determining whether said respective received input is within said predetermined parameter limits of said respective improvement exercise associated with at least one of said plurality of predetermined error types;
for each of said received inputs, associated with a respective improvement exercise, and responsive to said determination that said respective received input is within said predetermined parameter limits associated with said at least one of said plurality of predetermined error types, identifying said at least one of said plurality of predetermined error types;
for each of said received inputs, associated with a respective improvement exercise, and responsive to said error type identification, storing an indication of said identified at least one of said plurality of predetermined error types on said memory;
applying said predetermined first function to said indications of said identified error types for said received inputs associated with said respective output improvement exercises;
comparing an output of said applied predetermined first function, associated with said respective output improvement exercises, to each of said plurality of models stored on said memory;
responsive to an outcome of said comparison, associated with said respective output improvement exercises, identifying which of said plurality of field profiles describes the user;
comparing said identified field profile of the user, associated with said output improvement exercises, with said identified field profile of the user associated with said output diagnostic exercises; and
controlling said output device to output a respective portion of a respective set of said plurality of improvement exercises, said respective portion of said respective set responsive to an outcome of said comparison of said identified field profiles.

20. The method of claim 19, further comprising outputting an outcome of said comparison of said identified field profiles.

21-22. (canceled)
